(19) United States
(12) Patent Application Publication
(10) Pub. No.: US 2026/0207455 A1
(43) Pub. Date: Jul. 23, 2026

(54) ISOTROPIC LIQUID WASH COMPOSITION WITH ESTERIFIED NONIONIC THICKENER

(71) Applicant: Conopco, Inc., d/b/a UNILEVER, Hoboken, NJ (US)

(72) Inventors: Ashmiu Omarr Maadakin KOROMA, New Haven, CT (US); Tirucherai Varahan VASUDEVAN, Bethany, CT (US); Kimberly DAY, Orange, CT (US)

(73) Assignee: Conopco, Inc., d/b/a UNILEVER, Hoboken, NJ (US)

(21) Appl. No.: 19/143,322

(22) PCT Filed: Dec. 20, 2023
(86) PCT No.: PCT/EP2023/086972

§ 371 Date: Jun. 25, 2025

(30) Foreign Application Priority Data
Dec. 27, 2022 (EP) 22216807.2

(51) Int. Cl. A61K 8/46; A61K 8/37; A61K 8/44; A61Q 19/10

(52) U.S. Cl. CPC A61K 8/466; A61K 8/37; A61K 8/44; A61Q 19/10; A61K 2800/48

(57) ABSTRACT

Isotropic wash compositions with fatty acid esterified nonionic thickener are described. The wash compositions can be translucent or clear, are stable, mild and suitable to deliver benefit agents, like organic acids, for anti-acne benefits.

### FIELD OF THE INVENTION

The present invention is directed to a mild and stable translucent or transparent isotropic liquid wash composition. More particularly, the liquid wash composition comprises anionic and zwitterionic surfactants whereby the anionic surfactant is at least 45% by weight of a taurate, based on total weight of the anionic surfactant. The liquid wash composition has a pH of 5.25 or less, is suitable to deliver anti-microbial agents, including anti-acne agents, and can be made substantially free of at least one of sulfates, soaps, epoxides (like 1,4-dioxane), ethanol, halogenated antimicrobial actives, cyclosilicones, parabens, hydantoins and/or isothiazolinones.

### BACKGROUND OF THE INVENTION

Cleansing compositions, including bars and liquids, are known to provide benefits to consumers using them, and particularly, antimicrobial benefits. Microbes, like bacterium, are prevalent in our bodies and on the surface of our skin. Many consumers are surprised to hear that some bacterium and other microbes are essential for animal, including human, survival.

Our skin needs a certain amount of oil (i.e., sebum) to keep the skin healthy and soft. It is not uncommon, unfortunately, for the skin's sebaceous glands to produce more oil than is needed for skin to be healthy, especially and for example, when a consumer is stressed, subjected to hot climates and/or living in a city that has excessive pollution. It is well understood that not all bacterium is good, and excess oil on the skin is fuel for the common skin bacterium, Propionibacterium acnes (P. acnes), to feast on. When excess oil is present, it invariably leads to skin pore clogging, P. acnes proliferation, skin inflammation and immune responses that result in blemishes, like pustules or papules (pimples).

When skin health is compromised with blemishes, a consumer's self-esteem is often negatively impacted, especially with teens. While wash compositions can aid in minimizing the impact of excess sebum, traditional compositions often comprise harsh surfactants that strip too much sebum off the skin (making the skin dry and itchy) or mild surfactants that inefficiently clean. Moreover, such traditional compositions are overly thin or overly thick and opaque, resulting in poor cleansing and/or compositions that leave a residue or film that is difficult to wash off, and viewed negatively by consumers.

There is an increasing interest to develop mild and stable wash compositions that are gentle on skin, cleanse well and that have consumer desirable characteristics such as composition consistency, transparency or translucency, and the ability to rinse off easily and deliver benefit agents like anti-microbial agents.

This invention, therefore, is directed to a mild and stable translucent or transparent isotropic liquid wash composition. More particularly, the liquid wash composition comprises anionic and zwitterionic surfactants whereby the anionic surfactant is at least 45% by weight of a taurate based on total weight of the anionic surfactant. The liquid wash composition has a pH of 5.25 or less, is suitable to deliver anti-microbial agents, like anti-acne agents, and can be made substantially free of at least one of sulfates, soaps, epoxides (like 1,4-dioxane), ethanol, halogenated antimicrobial actives, parabens, hydantoins and/or isothiazolinones. Such a composition is unexpectedly mild, translucent or transparent and able to maintain excellent viscosity characteristics, even when formulated at a pH of 5.25 or less.

### Additional Information

Efforts have been disclosed for making antibacterial soap bars. In U.S. Pat. No. 6,794,344, soap based bar compositions having 50% soap with an alkyl chain length of 8 to 10 carbon atoms are described.

Other efforts have been disclosed for making soap-based formulations. In U.S. Patent Application Publication No. 2010/0098776, disclosed are soap-based liquid wash formulations with enhanced deposition of conditioning and/or skin appearance enhancing agents.

Even other efforts have been disclosed for making soap bar compositions. In WO16024090A1, a semi-translucent soap bar composition with antimicrobial benefit is described.

Still other efforts have been disclosed for making antimicrobial formulations. In U.S. Pat. No. 11,110,048, topical sanitizing formulations with quaternary ammonium salts are described.

Another effort has been disclosed for making anti-acne compositions. In U.S. Patent Application Publication No. 2007/0207112, anti-acne compositions with anti-microbial peptides are described.

None of the additional information describes a mild and stable translucent or transparent isotropic liquid wash composition as claimed herein.

### SUMMARY OF THE INVENTION

In a first aspect, the present invention is directed to an isotropic wash composition comprising:
a) an anionic surfactant comprising at least 75%, and preferably, from 80 to 100%, and most preferably, from 90 to 100% (or 95 to 100% or 100%) by weight of a taurate;
b) a zwitterionic surfactant comprising a betaine;
c) a C₁₄-C₂₀ (or preferably, a C₁₄-C₁₈, or most preferably, a C₁₆-C18) saturated or monounsaturated fatty acid esterified nonionic thickener, the nonionic thickener having a polyhydroxylated core with from 2 to 10, and preferably, 2 to 8, and most preferably, 2 to 6 (or 3, or 4, or 5) hydroxy groups and 10 to 100%, and preferably, from 20 to 80%, and most preferably, from 25 to 60% (or 25 to 55%) of total hydroxy groups on the nonionic thickener are esterified and when the nonionic thickener has 3 to 10 hydroxy groups, from 5 to 40%, and preferably, from 5 to 30%, and most preferably, from 5 to 20% (or 5 to 15% or 5 to 10%) of the total hydroxy groups are ether linked to polyethylene glycol (total ethylene glycol groups being from 5 to 300, and preferably, from 10 to 275, and most preferably, from 20 to 250 or 40 to 225); and
d) water,
   wherein the weight ratio of anionic surfactant to zwitterionic surfactant is from 1:3.5 to 3.5:1; and
   further wherein the wash composition:
i) is translucent or clear;
ii) optionally comprises an organic acid having a Log P from 1.0 to 8 or from 1.1 to or from 1.3 to 3.5;
iii) has a viscosity from 3,750 to 15,000 cps;
iv) has a pH from 3.75 to 5.25;
v) optionally has at least one carbon in the thickener that has carbon recovered from carbon capture; and
vi) optionally comprises an anionic surfactant which is a sulfate.

In a second aspect, the present invention is directed to an isotropic wash composition comprising:
a) an anionic surfactant comprising at least 75%, and preferably, from 80 to 100%, and most preferably, from 90 to 100% (or 95 to 100% or 100%) by weight of a taurate;
b) a zwitterionic surfactant comprising a betaine;
c) a C₁₄-C₂₀ (or preferably, a C₁₄-C₁₈, or most preferably, a C₁₆-C18) saturated or monounsaturated fatty acid esterified nonionic thickener, the nonionic thickener having a polyhydroxylated core with from 2 to 10, and preferably, 2 to 8, and most preferably, 2 to 6 (or 3, or 4, or 5) hydroxy groups and 10 to 100%, and preferably, from 20 to 80%, and most preferably, from 25 to 60% (or 25 to 55%) of total hydroxy groups on the nonionic thickener are esterified, and at least 8%, and preferably, at least 12%, and most preferably, from 15 to 100% (or from 15 to 55% or from 16 to 35% or from 18 to 30% or 100%) of total fatty acid forming the esterified nonionic thickener in the wash composition has saturated C₁₆-C18 fatty acid moiety, C₁₈:1, cis-9 fatty acid moiety or a mixture thereof; and
d) water,
   wherein the weight ratio of anionic surfactant to zwitterionic surfactant is from 1:3.5 to 3.5:1; and
   further wherein the wash composition:
   i) is translucent or clear;
   ii) optionally comprises an organic acid having a Log P from 1.0 to 8 or from 1.1 to 5 or from 1.3 to 3.5;
   iii) has a viscosity from 3,750 to 15,000 cps;
   iv) has a pH from 3.75 to 5.25;
   v) optionally has at least one carbon that has carbon recovered from carbon capture;
   vi) optionally comprises an anionic surfactant which is a sulfate.

In a third aspect, the present invention is directed to a method for washing with the isotropic wash compositions described in the first or second aspect of the invention.

In a fourth aspect, the present invention is directed to the use of a hydrophobically modified nonionic thickener having an esterified polyhydroxylated core to thicken wash compositions as described in the first two aspects of the invention where the wash compositions are isotropic, transparent and comprises an organic acid having a Log P from 1.0 to 8 or from 1.1 to 5 or from 1.3 to 3.5.

All other aspects of the present invention will more readily become apparent from the description and examples which follow.

Saturated or monounsaturated fatty acid esterified nonionic thickener means a thickener having at least 2 hydroxy groups on its center core (i.e., hydrophilic portion) esterified to such fatty acid, like for example polyethylene glycol-150 distearate. Skin, as used herein, is meant to include skin on the arms (including underarms), face, feet, neck, chest, hands, legs, back, buttocks and scalp (including hair). Wash composition, as used herein is meant to mean an isotropic composition, including those used to clean a topical surface such as the surface of an inanimate object, and preferably, skin. The wash composition of the present invention can be a shampoo, make-up remover, face cleanser or personal wash for body and/or hands. Such a wash composition is also suitable to use as a home care cleanser for an inanimate object, and therefore, a cleanser for an automobile, table, window, sink, counter, toilet or a cleanser for laundry or upholstery. In an embodiment of the invention, the wash composition is a personal wash composition for skin. In another embodiment, the wash composition is a personal wash composition for the hands. In even another embodiment, the wash composition is a personal wash composition for the face, and preferably, a face wash that delivers (and is marketed for) anti-microbial benefits like anti-acne benefits. The wash composition typically includes 50 to 90%, and preferably, 55 to 85%, and most preferably, from 60 to 80% (or 60 to 75% or 62 to 72%) by weight water and has a pH from 3.75 to 5.25, and preferably, from 3.85 to 5.15, and most preferably, from 4.2 to 5.0 (or from 4.2 to 4.85). The wash composition is suitable to deliver an active pharmaceutical ingredient (e.g., like corticoids, antihistamines and/or antibiotics). In a preferred embodiment, however, the wash composition is a cosmetic and non-therapeutic composition suitable to remove soil (water and fat soluble) and suppress and remove bacteria from topical surfaces, and preferably, skin, and especially, skin on the hands and face. The wash composition is surprisingly mild, stable and translucent or transparent even when formulated to have a pH that does not exceed 5.25. Mild, as used herein, means trained panellists using the wash composition of the invention on their hands and face repeatedly and at least four (4) times in a day observed no skin irritation, as well as having a Zein value ≤7 as defined and determined by a method like the DB-ALM Protocol n° 26 Zein Test Method. Anti-acne benefits means trained panellists using the wash composition of the invention on their faces repeatedly and at least two (2) times per day for one (1) week visually observed a reduction in acne. In an embodiment of the invention, anti-acne benefits is consistent with the definition in Title 21 of the Code of Federal Regulations. log P is the log₁₀ (Partition Coefficient) where the partition coefficient, P is equal to [Compound]_{octanol}/[Compound]_{water}.

Substantially free of (excluding dioxanes, soaps and sulfates) means no more than 2.0% by weight of the total weight of the wash composition, and preferably, less than 1.5% by weight of the total weight of the wash composition. In still another embodiment, substantially free of means less than 1%, and preferably, less than 0.5% by weight of the total weight of the wash composition. Substantially free of, as used herein, is meant to include 0.00001% to 2.0%, or 0.00001 to 1%, or 0.00001 to 0.5%, or 0.0% by weight of the total weight of the wash composition. As to soap, substantially free of (i.e., soap) means less than 5.5%, and preferably, less than 4.5%, and most preferably, less than 3.75% by weight soap based on total weight of the wash composition. In an embodiment of the invention, the wash composition comprises from 0.00001 to 3.5% or 0.001 to 3.35% by weight soap. In another embodiment, the liquid composition comprises no (0.0% by weight) soap. Betaine (or sultaine) as used herein is meant to mean a zwitterionic surfactant derived from the same. In the case of betaine, for example, a preferred surfactant derived therefrom includes cocoamidopropyl betaine.

In still another embodiment of the invention, the wash composition can be formulated to be substantially free of sulfates, and means, can optionally up to 5% by weight of anionic sulfate comprising surfactant, and preferably, less than 3.5%, and most preferably, less than 1.5% by weight sulfate (or less than 1% or less than 0.5% or from 0.0001 to 0.5% or no (0.0%) sulfate. In yet another embodiment of the invention, the wash composition comprises less than 25 ppm or less than 10 ppm or less than 3 ppm 1,4-dioxane (or less than 2 ppm or less than 1 ppm or from or from 0.0 to 0.5 ppm). In still another embodiment, the wash composition less than 2% by weight silicone or 0.0% by weight silicone (e.g., no cyclosiloxane like decamethylcyclopentasiloxane).

Isotropic wash is defined to mean a composition with micelles arranged without directional preference. Transparent means clear and able to see through, Translucent means not opaque and permitting light to pass with diffusion. Skin benefit agents can optionally be used in the liquid composition of the present invention and such agents may be water or oil soluble. The wash composition will have a viscosity from 3,750 to 22,000 cps as taken with a Brookfield helipath TD, with RV Spindle 5, at 25° C. and 20 rpm for 1 minute. In the absence of explicitly stating otherwise, all ranges described herein are meant to include all ranges subsumed therein. The term comprising is meant to encompass the terms consisting essentially of and consisting of. For the avoidance of doubt, and for illustration, a composition comprising water, anionic surfactant and acid is meant to include a composition consisting essentially of the same and a solution consisting of the same. Except in the operating comparative examples, or where otherwise explicitly indicated, all numbers in this description indicating amounts or ratios of materials or conditions and/or physical properties of materials and/or use are to be understood as modified by the word “about”.

### DETAILED DESCRIPTION OF THE INVENTION

As to the anionic surfactant used in the isotropic wash composition of the present invention, at least 75% of the total weight of anionic surfactant is a taurate (or salt thereof). Illustrative examples of the taurate surfactants suitable to be used includes those which are acylamides of taurine or N-methyltaurine, and salts thereof. For example, taurates suitable for use are acyl taurates represented by the general formulae:

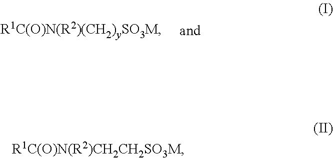

where R¹ is C₆ to C₃₀, more particularly, C₆ to C₂₄ alkyl, y is 2 or 3, R² is hydrogen or methyl, and M is hydrogen or a solubilizing cation such as, for example, hydrogen, ammonium, alkali metal cation, a lower C₁ to C₄, alkanol ammonium cation and/or a basic amino acid cation. In one embodiment, R¹ is C₈ to C18 alkyl. In another embodiment at least half of the R¹ groups are C₈-C18 alkyl. In still another embodiment at least half of the R¹ groups are C₁₀ to C₁₄ alkyl. R¹ may be saturated or unsaturated. In yet another embodiment R² is methyl.

In an embodiment of the invention, the acyl taurates selected for use in the invention include, for example, taurates commonly known as sodium methyl lauroyl taurate, sodium methyl myristoyl taurate, sodium methyl cocoyl taurate, sodium methyl oleoyl taurate, sodium cocoyl taurate, potassium methyl myristoyl taurate, calcium methyl lauroyl taurate, potassium methyl lauroyl taurate, ammonium methyl lauroyl taurate or a mixture thereof. In an embodiment of the invention, the preferred taurate used is sodium methyl lauroyl taurate.

As to optional anionic surfactants that may be included at less than 25% by weight of the total weight of anionic surfactant used, these include glycinates and salts of the same. Illustrative examples of the glycinates suitable for optional use include those having the formula:

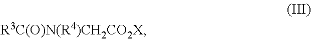

where R³ is a C₈-C₂₄ (preferably, a C₁₀-C₂₄) alkyl group, R⁴ is H or a CH₃ and X is hydrogen a cation such as sodium, potassium, ammonium, a triethanolammonium ion or a mixture thereof.

Illustrative examples of the often-preferred optional glycinates suitable for use in the wash composition include sodium cocoyl glycinate, potassium cocoyl glycinate, sodium lauroyl glycinate, potassium lauroyl glycinate, sodium myristoyl glycinate, potassium myristoyl glycinate or a mixture thereof.

Glutamates and aspartates are yet another type of optional anionic surfactants suitable for use in the wash composition. Illustrative examples of glutamates suitable for use in the present invention include sodium lauroyl glutamate, sodium cocoyl glutamate, potassium lauroyl glutamate, potassium cocoyl glutamate, disodium cocoyl glutamate, mixtures thereof or the like. Illustrative examples of aspartates suitable for use either alone or together with glutamates and/or the anionic taurates include sodium lauroyl aspartate, potassium lauroyl aspartate, sodium cocoyl aspartate, potassium cocoyl aspartate, mixtures thereof or the like.

In even another embodiment of the invention, optional anionic surfactant suitable for use includes sulfate-based surfactants whereby the same can be linear or branched. Suitable examples of the linear anionic sulfate-based surfactants that may optionally be used include ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium laureth sulfate, sodium pareth sulfate, potassium laureth sulfate, ammonium cocoyl sulfate, ammonium lauroyl sulfate, sodium cocoyl sulfate, sodium lauryl sulfate, potassium cocoyl sulfate, potassium lauryl sulfate, monoethanolamine cocoyl sulfate, mixtures thereof or the like.

Illustrative branched sulfates suitable for optional use include sodium trideceth sulfate, sodium tridecyl sulfate, sodium C₁₂₋₁₈ alkyl sulfate (preferably C₁₄₋₁₈ or C₁₄₋₁₆ sodium alkyl sulfate) sodium C₁₂₋₁₆ pareth sulfate, sodium C₁₂₋₁₆ pareth-n sulfate, mixtures thereof or the like.

In an embodiment of the invention, additional salts of the aforementioned anionic sulfates can be used in lieu of or in addition to sodium salts, including triethanolamine, diethanolamine, ammonia, potassium and/or calcium salts. In even another optional embodiment of the invention, the triethanolamine salt of lauryl sulfate is used. In yet another embodiment of the invention, the anionic surfactant includes sodium lauryl sulfate, sodium pareth sulfate, sodium laureth sulfate or a mixture thereof. In still another embodiment, and as noted herein, the wash composition is substantially free of sulfate-based surfactants.

Alkyl sulfosuccinates and alpha-olefin sulphonates are also suitable for optional use as anionic surfactants. Alkyl sulfosuccinates that may be used include mono- and dialkyl (e.g., C₆-C₂₂ alkyl) sulfosuccinates. In an embodiment of the invention, the sulfosuccinates suitable for use include sodium dioctyl sulfosuccinate, dihexyl sulfosuccinate, sodium dicyclohexyl sulfosuccinate, disodium laureth sulfosuccinate, disodium lauryl sulfosuccinate disodium isodecyl sulfosuccinate mixtures thereof or the like. Commercially available sulfosuccinates are sold by suppliers like Cytec Solvay Group, Stepan Company and Dow Chemical Company. In an embodiment of the invention, C₃-C₂₀ alkyl sulfosuccinates and/or C₁₀-C18 alkyl sulfosuccinates are selected for use. In still another embodiment, disodium lauryl sulfosuccinate is the anionic surfactant included.

In another embodiment, the alpha-olefin sulphonate suitable for optional use is typically a C₁₂-C18, and preferably, a C₁₂-C₁₆, and most preferably, sodium C₁₄-C₁₆ alpha-olefin sulphonate or mixtures thereof including those made commercially available by Stepan Company and Clariant.

Alkyl benzene sulfonates like sodium tridecyl benzene sulfonate, sodium dodecyl benzene sulfonate may also be considered for use.

The wash composition of the present invention can optionally comprise an isethionate as anionic surfactant at levels that do not exceed 1% by weight of the wash composition. Preferably, when used, isethionate is present at from 0.001 to 0.85%, or from 0.001 to 0.5% by weight of the wash composition. In an embodiment of the invention, the isethionate used is sodium lauroyl isethionate, sodium cocoyl isethionate, and preferably, sodium 1-methyl isethionate or a mixture thereof. In yet a preferred embodiment, no isethionate is used (0.0% by weight) or only an acyl 1-alkyl, 2-alkyl and/or 1,2-alkyl isethionate where the alkyl group is often a C₁-C₄-alkyl, and preferably, a C₁ and/or C₂ alkyl, and most preferably, a methyl group. If an isethionate, sodium 1-methyl isethionate is preferably selected for use.

As to zwitterionic surfactants used in the compositions of the present invention, such surfactants include at least one acid group. Such an acid group may be a carboxylic or a sulphonic acid group. They often include quaternary nitrogen, and therefore, can be quaternary amino acids. They often generally include an alkyl or alkenyl group of 7 to 18 carbon atoms and generally comply with an overall structural formula:

R⁵—[—C(O)—NH(CH₂)_{q}—]ᵣ—N⁺—(R⁶—)(R⁷)A-B where R⁵ is alkyl or alkenyl of 7 to 18 carbon atoms; R⁶ and R⁷ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂- or —SO₃—.

Preferred zwitterionic surfactants used in the present invention include are betaines of the formula:

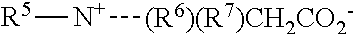

and amido betaines of the formula:

R⁵—CONH(CH₂)ₜ—N⁺—(R⁶(R⁷)CH₂CO₂⁻ where t is 2 or 3.

In both formulae R⁵, R⁶ and R⁷ are as defined previously. R⁶ may, in particular, be a mixture of C₁₂ and C₁₄ alkyl groups derived from coconut oil so that at least half, preferably at least three quarters of the groups R⁶ have 10 to 14 carbon atoms. R⁶ and R⁷ are preferably methyl.

Another option is that the zwitterionic surfactant is a sulphobetaine of the formula:

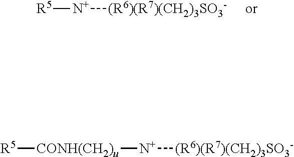

where u is 2 or 3, or variants of these in which —(CH₂)₃SO₃⁻ is replaced by —CH₂C(OH)(H)CH₂SO₃⁻.

In these formulae, R⁵, R⁶ and R⁷ are as previously defined.

Illustrative examples of the zwitterionic surfactants suitable for use include betaines like coco betaine, cocamidopropyl betaine, lauryl betaine, laurylhydroxy sulfobetaine, lauryldimethyl betaine, cocamidopropylhydroxylsulfo betaine, behenyl betaine, capryl/capramidopropyl betaine, cocodimethyl carboxymethyl betaine, stearyl betaine.
laurylamidopropyl betaine, mixtures thereof or the like. An additional zwitterionic surfactant suitable for use includes lauryl hydroxysultaine and/or cocamidopropyl sultaine. In a preferred embodiment, the zwitterionic surfactant used in the wash composition of this invention is cocamidopropyl betaine.

In still another embodiment of the invention, anionic surfactant and zwitterionic surfactant are used at a weight ratio of anionic to zwitterionic surfactant 1:4.5 to 4.5:1 or from 3.5:1 to 1:3.5 or from 1:3 to 3:1 or from 2.75:1 to 1:2.75. to 2.65:1. In even another embodiment of the invention, the anionic surfactant to zwitterionic surfactant are at a weight ratio of 1:2.65 or from 1:2.5 or from 1:2.3 or from 1:2.15. In another embodiment of the invention, the anionic surfactant used is at least 95% or 100% by weight of a taurate where sodium lauroyl methyl taurate is the taurate preferred. In even another embodiment of the invention, the zwitterionic surfactant makes is 70 to 100% or 80 to 100% or 90 to 100% by weight betaine or sultaine, preferably betaine. In yet another embodiment of the invention, the zwitterionic surfactant used is at least 95% to 100% by weight of a betaine where cocamidopropyl betaine is the preferred betaine.

In still another embodiment, anionic surfactant and zwitterionic surfactant make up at least 85%, and preferably at least 90%, and most preferably, at least 95 to 99% or 96 to 99% or 100% by weight of the total weight of surfactant in the wash composition.

While not required, amphoteric surfactants can optionally be included like sodium lauroamphoacetate, sodium cocoamphoacetate, potassium lauroamphoacetate, potassium cocoamphoacetate or mixtures thereof. If used, amphoteric surfactant will make up from 0.001 to 1.65% by weight of the wash composition.

Other optional surfactants suitable for use include cationic surfactants like heterocyclic ammonium salts such as cetyl or stearyl pyridinium chloride, alkyl amidoethyl pyrrylinodium methyl sulfate, lapyrium chloride or a mixture thereof. If used, cationic surfactants typically do not exceed 1.5% by weight of the wash composition.

Nonionic surfactants are another optional surfactant suitable for use in the wash composition of the invention, and they include polyglycerol esters like polyglyceryl-8 caprylate, polyglycerol-8 caprate, polyglyceryl-8 myristate, polyglyceryl-8 palmitate, polyglyceryl-9 caprylate, polyglycerol-9 caprate, polyglyceryl-9 laurate, polyglyceryl-9 myristate, polyglyceryl-9 palmitate, cocamide monoethanolamine (Cocamide MEA), Polysorbate 20 (Tween 20) or a mixture thereof. If used, nonionic surfactant typically makes up from 0.001 to 2.5%, and preferably, from 0.0 to 1.75%, and most preferably from 0.1 to 0.8% by weight of the wash composition. When used, cocamide monoethanolamine and/or Tween 20 is/are particularly preferred.

The wash compositions of the present invention will typically not have more than 15% by weight total surfactant, and preferably, from 7 to 14.5%, and most preferably, from 8 to 13% or from 9 to 12% or from 9.5 to 11.5% total surfactant. Often, zwitterionic surfactant makes up from 5 to 9%, and preferably, from 6 to 9%, and most preferably, from 6 to 8% by weight of the wash composition and anionic surfactant makes up from about 2.5 to 5%, and preferably, from 3 to 5%, and most preferably, from 3.2 to 4.5% by weight of the wash composition.

As to the organic acids suitable for optional use, these include acids having a log P from 0.0 to 4.95 or from 1.0 to 4.5 or from 1.3 to 3.5. Illustrative examples of the acids suitable for use include coumaric, benzoic, p-amino benzoic, salicylic, capryloyl salicylic, 3-hydroxybenzoic, 4-hydroxybenzoic and/or gallic acid. Phosphonic acids suitable for use include 4-ethoxyphenylphosphonic, 4-methylphenylphosphonic, 4-chlorophenylphosphonic, phenylphosphonic acid. In an embodiment of the invention, benzoic, p-amino benzoic, salicylic, capryloyl salicylic acid or a mixture thereof is used.

Other suitable and optional organic acids that may be used include carboxylic acids like propionic, n-butryic, isobutryic, pentanoic, hexanoic, heptanoic, octanoic, nonanoic and/or decanoic acid. Dicarboxylic acids suitable for use include adipic and/or pimelic acid. The carboxylic acids can also include but-3-enoic, but-2-ynoic, crotonic, isocrotonic, sorbic, angelic, tiglic, tetrolic acid or mixtures thereof.

When used, organic acid (including mixtures of the same) makes up from 0.5 to 2.5% or from 0.7 to 2.2% or from 0.8 to 2.0% or from 0.8 to 1.6% by weight of the wash composition. In an embodiment of the invention, the acid used is benzoic, p-aminobenzoic, capryloyl salicylic acid and/or salicylic acid. In another embodiment, the acid used comprises salicylic acid and benzoic acid at a weight ratio of 5:95 to 95:5, and preferably, from 25:75 to 75:25, and most preferably, from 40:60 to 60:40. In another embodiment of the invention, the acid used consists essentially of salicylic acid and benzoic acid at a weight ratio of 5:95 to 95:5, and preferably, from 25:75 to 75:25, and most preferably, from 40:60 to 60:40. In even another embodiment of the invention, the acid used is all salicylic acid or all benzoic acid (i.e., 100% by weight of the total weight of acid used).

As to the hydrophobically modified nonionic thickener, the same typically makes up from 0.03 to 1.5, or from 0.04 to 1.25% or from 0.05 to 1.0% or from 0.06 to 0.9% or from 0.07 to 0.2% or from 0.08 to 0.13% by weight of the wash composition. In an embodiment of the invention, the preferred hydrophobically modified nonionic thickener is polyethylene glycol-150 distearate, or polyethylene glycol-120 methyl glucose trioleate sold as Glucamate™ VLT and made available by Lubrizol, or polyethylene glycol-200 hydrogenated glyceryl palmate and with polyethylene glycol-7 glyceryl cocoate as carrier sold as Antil® 200 made available by Evonik, or a mixture thereof.

Optional water dispersible polymers may optionally be used in the wash compositions of the present invention at levels that do not exceed 0.1%, or 0.05%, or 0.03% or 0.02% by weight of the wash composition. Examples include carbohydrate gums such as cellulose gum, microcrystalline cellulose, cellulose gel, hydroxyethyl cellulose, hydroxypropyl cellulose, sodium carboxymethylcellulose, hydroxymethyl or carboxymethyl cellulose, methyl cellulose, ethyl cellulose, guar gum, gum karaya, gum tragacanth, gum Arabic, gum acacia, gum agar, xanthan gum and mixtures thereof; modified and nonmodified starch granules with gelatinization temperatures between 30 to 85° C. and pregelatinized cold water soluble starch; polyacrylate; Carbopols; alkaline soluble emulsion polymer such as Aculyn 28, Acuyln 22 or Carbopol Aqua SF1; cationic polymer such as modified polysaccharides including cationic guar available from Rhone Poulenc under the trade name Jaguar C₁₃S, Jaguar C₁₄S, Jaguar C₁₇, or Jaguar C₁₆; cationic modified cellulose such as UCARE Polymer JR 30 or JR 40 from Amerchol; N-Hance 3000, N-Hance 3196, N-Hance GPX 215 or N-Hance GPX 196 from Hercules; synthetic cationic polymer such as MerQuat 100, MerQuat 280, Merquat 281 and Merquat 550 by Nalco; cationic starches, e.g., StaLok® 100, 200, 300 and 400 made by Galactasol 800 series by Henkel, Inc.; Quadrosoft Um-200; and Polyquaternium-24. In an embodiment of the invention, from 0.001 to 0.02% by weight of such thickener is used. In a preferred embodiment, none of such water dispersable polymers, 0.0% by weight of such polymers are used in the wash compositions of the present invention. In another embodiment of the invention, 0.0% by weight of an acrylate based thickener, including those referred to as a Carbopol®, Aculyn™, or Ultrez 10, 20 or 21.

Fatty acid may optionally be included in the liquid composition of the present invention, the same is often a blend of C to C18, preferably C₁₂ to C18, and most preferably, C₁₆ to C₁₈ fatty acid. Especially preferred is palmitic and/or stearic acid, and most preferably, stearic acid. Typically, when used, such acids will make up from 0.02 to 1.5%, and preferably, from 0.03 to 1.35%, and most preferably, from 0.05 to 1.25% by weight of the translucent or transparent isotropic wash composition.

Additional fatty acids suitable to optionally be included are unsaturated fatty acids like linoleic acid (C18:2), oleic acid (C18:1) or mixtures thereof. In an embodiment of the invention, when unsaturated fatty acid is used, the same makes up less than 1%, and preferably, less than 0.75%, and most preferably, less than 0.5% by weight of the total weight of the wash composition. In another embodiment, unsaturated fatty acid makes up from 0.0001 to 0.5% by weight of the total weight of the wash composition. In another embodiment of the invention and when unsaturated fatty acid is used, preferably less than 25% of the unsaturated fatty acid used is oleic acid.

Regarding soaps, and as noted, the wash compositions of the present invention are substantially free of soap. If optionally used in the wash composition of the present invention, the same can include salts that are C to C₁₄ and/or C₁₆ to C₁₈ soaps including sodium, potassium or ammonium soaps of lauric, palmitic or stearic acid (laurate, palmitate and/or stearate soap). The transparent or translucent isotropic wash compositions of the present invention will preferably have 0.0% by weight soap.

Water typically makes up from 35 to 85%, and preferably, from 45 to 80%, and most preferably, from 55 to 75% by weight of the wash composition.

The wash composition of the present invention will have a viscosity from 3,750 to 15,000 cps, and preferably, from 3,800 to 20,000 cps, and most preferably, from 3,900 to 19,000 cps and even more preferably, from 4,000 to 18,500 cps, and whereby 1 cps (centipoise) equals 1 mPa·s (millipascal-second). In even another embodiment of the invention, the wash composition has a viscosity from 5,000 to 14,000 cps or from 14,000 cps to 19,000 cps.

Optional but preferred ingredients that may be used in the translucent or transparent isotropic wash compositions of the present invention include preservatives to assist against the growth of potentially harmful microorganisms when the end use wash composition is made. Suitable traditional preservatives that may be used include propionate salts, and a variety of quaternary ammonium compounds. Often preferred preservatives are potassium sorbate, iodopropynyl butyl carbamate, phenoxyethanol, wasabi-based preservatives, imidazolidinyl urea, sodium dehydroacetate and benzyl alcohol. Often desired additives suitable to be employed in the liquid composition of the present invention are 1,2-alkanediols like 1,2-octanediol, 1,2 hexanediol or mixtures thereof. The liquid composition may comprise of a preservative system that is formaldehyde-free, paraben-free, or both. In an embodiment of the invention the preservative used is sodium and/or potassium benzoate whereby preservatives that are isothiazolinones are preferably not used, and therefore, make up less than 0.2% by weight of the wash composition, and more preferably, 0.0% by weight of the wash composition.

When employed, the preservatives, and/or vicinal diol component will not make up collectively more than 2%, and preferably, not more than 1.3%, and most preferably, from 0.2 to 1.2% by weight of the wash composition. In an embodiment of this invention, from 0.2 to 1.0% by weight preservative, and/or vicinal diol component is used, based on total weight of the wash composition.

Fragrance components like eugenol, coumarin, linalyl acetate, citronellal, iris concentrate, terpinyl acetate, terpineol, thymol, pinenes (e.g., alpha and beta pinene) and citronellol may optionally be added to the wash composition. Such components make up from 0.01 to 1.2%, and preferably, from 0.1 to 1%, and most preferably, from 0.1 to 0.65% by weight of the liquid composition when used.

Other optional ingredients suitable for use include zinc pyrithione, octopirox, or a mixture thereof, especially when the liquid composition is used as a shampoo that provides antidandruff benefits. Each of these substances may range from 0.05 to 3%, and preferably, between 0.1 and 2% by weight of the total weight of the wash composition.

Additional optional ingredients that may be used include sensory oils and/or exfoliants. Desirable oils include rose, lime, coconut, lavender, argan, tea tree, sweet almond oil(s) or mixtures thereof. Illustrative exfoliants desirable for use include salt, sugar, apricot, walnut shell, rice, nutmeg and/or oatmeal powder(s). When used, sensory oils and exfoliants can make up from 0.1 to 2% by weight of wash composition, with the proviso that the total amount of fragrance and sensory oil does not exceed 2.5% by weight of the composition, and preferably, not more than 2.0 percent by weight of the wash composition.

The wash composition may optionally include vitamins. Illustrative vitamins include Vitamin B₂, Vitamin B₃ (niacinamide), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of optional vitamins when present may range from 0.001 to 5%, and preferably from 0.01% to 3%, optimally from 0.1 to 1% by weight of the wash composition.

Other optional additives suitable for use include resorcinols like 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol, thiamidol; alpha- and/or beta-hydroxyacids; retinoic acid and its derivatives (e.g., cis and trans); retinal; retinol; retinyl esters such as retinyl acetate, retinyl palmitate, and retinyl propionate; petroselinic acid; conjugated linoleic acid; 12-hydroxystearic acid; mixtures thereof or the like. Conditioning agents such as polyquaternium compounds (e.g., polyquaternium-67) may also be desirable for inclusion in the inventive composition. Such additives, when used, collectively make up from 0.001 to 3%, preferably, from 0.01 to 2%, and, most preferably, from 0.1 to 1.5% by weight of the wash composition.

Desquamation promoters may be present. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Amounts of these materials when present may range from 0.01 to 3%, and, preferably, from 0.1 to 2% by weight of the wash composition.

A variety of herbal extracts may optionally be included in the cosmetic wash compositions of this invention. Illustrative extracts include those removed from green tea, yarrow, ginseng, marigold, hibiscus, ginko biloba, chamomile, licorice, aloe vera, grape seed, citrus unshiu, willow bark, sage, rosemary and bambusa vulgaris shoot extract. When used, extracts make up from 0.001 to 1%, and preferably from 0.01 to 0.8%, and most preferably, from 0.03 to 0.5% by weight of the wash composition.

Humectants like glycerol and other polyols (e.g., sorbitol) may also be included. Humectants preferably glycerine, when used, typically make up from 0.01 to 8%, preferably, from 0.01 to 6%, and most preferably, from 0.02 to 4% by weight of the wash composition.

Another optional additive suitable for use includes hemp oil with 2.5 to 25% by weight cannabigerol and/or cannabidiol at from 0.5 to 10 percent by weight. When used, such oil makes up from 0.0001 to 5% by weight of the composition, and preferably, from 0.01 to 3% by weight of the wash composition.

Also, optionally suitable for use include materials like chelators (e.g., EDTA), opacifiers (like TiO₂, particle size from 50 to 1200 nm, and preferably, 50 to 350 nm), kaolin, bentonite, zinc oxide, iron oxide, mica, C₈₋₂₂ fatty acid substituted saccharides, lipoic acid, retinoxytrimethylsilane (available from Clariant Corp. under the Silcare 1M-75 trademark), dehydroepiandrosterone (DHEA) or mixtures thereof. Ceramides (including Ceramide 1, Ceramide 3, Ceramide 3B and Ceramide 6) as well as pseudoceramides may also be optionally included as can 10-hydroxystearic acid. Amounts of these additional and optional materials, when used, may range from 0.0001 to 3%, and preferably, from 0.001 to 2%, and most preferably, from 0.001 to 1.5% by weight of the wash composition.

Colorants or dyes may also be included in the wash compositions. These substances may range from 0.05 to 5%, and preferably, between 0.1 to 2% by weight of the wash composition.

Conditioning agents like hydroxypropyltrimonium chloride, 5-ureidohydantoin and/or glyoxyldiureide may be used. Such components, when used, make up from 0.5 to 4%, and, preferably, from 0.75 to 3%, and most preferably, from 1 to 2% by weight of the wash composition.

Sunscreen actives may also be optionally included in the wash composition of the present invention. Particularly preferred are such materials as ethylhexyl p-methoxycinnamate, available as Parsol MCX®, Avobenzene, available as Parsol 1789© and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.01 to 3%, and preferably, from 0.5 to 2%, optimally, from 0.75 to 1.5% by weight of the wash composition.

Conventional buffers/pH modifiers may be used. These include commonly employed additives like sodium hydroxide, potassium hydroxide, hydrochloric acid, citric acid/citrate buffers, triethanolamine, or mixtures thereof where pH is determined using a Thermo Fisher Scientific pH meter.

Optional antimicrobial ingredients may be used but are not required. Illustrative ingredients include quaternary ammonium compounds like cetylpyridinium chloride, cetyltrimethylammonium chloride, cetyltrimethylammonium bromide, benzalkonium chloride, benzethonium chloride, cetrimide or mixtures thereof. Cetrimonium chloride and/or bromide, chloroxylenol, antimicrobial lipids, antimicrobial peptides or mixtures thereof may also be used. Triclosan may also be used but is not generally desired. Mixtures of thymol and terpineol are often desired for use, typically at a weight ratio from 1:3 to 3:1 or from 1:2 or 2:1 or from 1:0.7 to 0.7:1. When used, such optional antimicrobial ingredients make up from 0.001 to 6%, and preferably, from 0.01 to 5%, and most preferably, from 0.01 to 2.5% by weight of the wash composition.

When preparing wash composition, conventional techniques may be used, and these include moderate shear mixing at atmospheric pressure and temperatures between 25 to 50° C.

A wide variety of packaging can be employed with the wash composition of this invention. Laminated boxes and bags as well as plastic containers may be used. Preferably, the packaging used with the composition of the present invention is packaging made from recycled material like post-consumer resins or biodegradable material. Most preferably, the wash composition is sold in biodegradable packaging material, like post-consumer resins.

The following examples are provided to facilitate an understanding of the present invention. The examples are not intended to limit the scope of the claims.

### Example 1

The ingredients used to make the Samples of Table I were mixed with moderate shear under atmospheric conditions and with temperature at 35° C. The Samples were made according to the present invention whereby hydrophobically modified nonionic thickener was used with a betaine to taurate weight ratio of 1.9:1. Viscosity was measured with a Brookfield Viscometer as described herein. The ingredients are presented as weight percent (Wt. %) based on total weight of the wash composition. Water was added to balance at 100%.

TABLE I

| | Sample I | Sample II |
|---|---|---|
| Ingredient | Wt. % | Wt. % |
| Water | q.s | q.s |
| PEG-150 Distearateⁱ | 0.1 | 0.3 |
| Fragrance | 0.75 | 0.75 |
| Sodium Methyl Lauroyl Taurate | 3.6 | 3.6 |
| Cocamidopropyl Betaine | 6.9 | 6.9 |
| Glycerin | 1.5 | 1.5 |
| Sodium Benzoate | 0.2 | 0.2 |
| Salicylic Acid | 1 | 1 |
| CMEAⁱⁱ | 0.68 | 0.68 |
| pH | 4.27 | 4.27 |
| Viscosity (4 1/s) | 11840 | 16560 |
| Viscosity (10 1/s) | 10780 | 14510 |

ⁱPolyethylene glycol-150 distearate, and the composition may be made with Glutamate VLT and/or Antil-200 with polyethylene glycol-150 distearate or in lieu of such thickener.
ⁱⁱCocamide monoethanolamine

The compositions (Samples I-II) of Example 1 were made consistent with the present invention and were made free of (0.0% by weight) sulfates, soaps, epoxides, ethanol, halogenated antimicrobial actives, cyclosilicones, parabens, hydantoins and isothiazolinones yet surprisingly remained stable with no syneresis and maintained favorable viscosities. Moreover, after being used by trained panelists, it was unexpectedly concluded that the composition provided excellent sensory characteristics, were mild on the face and remained very clear (i.e., transparent) even when formulated with 10.5% by weight surfactant, anti-acne ingredient (organic acid) and at a pH of 4.3.

### Example II

Samples Ill-VI were made in a manner consistent with the one described in Example I except hydrophobically modified nonionic thickener was not used according to the present invention.

TABLE II

| | Sample | Sample | Sample | Sample |
|---|---|---|---|---|
| | III | IV | V | VII |
| Ingredients | Wt. % | Wt. % | Wt. % | Wt. % |
| Aculyn ™ 33Aⁱ | — | — | 0.8 | 1.5 |
| SF-1ⁱⁱ | 0.8 | 1.5 | — | — |
| Fragrance | 0.75 | 0.75 | 0.75 | 0.75 |
| Sodium Methyl Lauroyl | 3.6 | 3.6 | 3.6 | 3.6 |
| Taurate | | | | |
| Cocamidopropyl Betaine | 6.9 | 6.9 | 6.9 | 6.9 |
| Glycerin | 1.5 | 1.5 | 1.5 | 1.5 |
| Sodium Benzoate | 0.2 | 0.2 | 0.2 | 0.2 |
| Salicylic Acid | 1 | 1 | 1 | 1 |
| CMEAⁱⁱⁱ | 0.68 | 0.68 | 0.68 | 0.68 |
| pH | 4.27 | 4.27 | 4.27 | 4.27 |
| Viscosity (4 1/s) | 6194 | 7931 | 6206 | 17710 |
| Viscosity (10 1/s) | 5605 | 6712 | 5327 | 13160 |

ⁱacrylate copolymer, Dow Chemical
ⁱⁱSF-1, Carbopol ® Aqua, Lubrizol, acrylate copolymer
ⁱⁱⁱCocamide monoethanolamine

The wash compositions made according to Example 2 did not comprise hydrophobically modified nonionic thickener consistent with the present invention. After being reviewed by skilled panelists, the compositions were not usable, were unstable, showed visual evidence of syneresis and/or ingredient flocculation and/or agglomerate formation. Additionally, the compositions made according to Example II were not clear but opaque, cloudy, not desirable for use and not homogeneous in appearance.

1.-11. (canceled)

12. An isotropic wash composition comprising:
a) an anionic surfactant, the anionic surfactant comprising from 80 to 100% by weight of a taurate;
b) a zwitterionic surfactant comprising a betaine;
c) 0.03 to 1.5% by weight of a saturated or monounsaturated fatty acid esterified nonionic thickener, the nonionic thickener having a polyhydroxylated core with from 2 to 10 hydroxy groups, and 10 to 100% of total hydroxy groups on the nonionic thickener are esterified and when the nonionic thickener has 3 to 10 hydroxy groups, from 5 to 40% of the total hydroxy groups are ether linked to polyethylene glycol with total ethylene glycol groups being from 5 to 300; and
d) water,
wherein the weight ratio of anionic surfactant to zwitterionic surfactant is from 1:4.5 to 4.5:1; and further wherein the wash composition:
i) is translucent or clear;
ii) comprises from 0.5 to 2.5% by weight of an organic acid having a Log P from 1.0 to 8 and comprising benzoic acid, p-aminobenzoic acid, capryloyl salicylic acid, salicylic acid or a mixture thereof;
iii) has a viscosity from 3,750 to 15,000 cps;
iv) has a pH from 3.75 to 5.25;
v) optionally has at least one carbon in the thickener that has carbon recovered from carbon capture;
vi) has less than 1% by weight of an anionic sulfate comprising surfactant; and
vii) comprises from 0 to 1.75% by weight nonionic surfactant and from 0.0 to 0.5% by weight acyl isethionate.

13. The isotropic wash composition according to claim 12 wherein the wash composition is clear, is sulfate comprising surfactant free and comprises an organic acid having a log P from 0.0 to 4.95.

14. The isotropic wash composition according to claim 12 wherein the wash composition comprises an organic acid having a log P from 1.0 to 4.5.

15. The isotropic wash composition according to claim 12 wherein the organic acid is salicylic acid, benzoic acid or a mixture thereof.

16. The isotropic wash composition according to claim 12 wherein the wash composition comprises thiamidol, niacinamide, a resorcinol, 12-hydroxystearic acid, a retinyl ester or mixture thereof.

17. The isotropic wash composition according to claim 12 wherein the thickener is polyethylene glycol-150 distearate, or polyethylene ethylene glycol-120 methyl glucose trioleate, or polyethylene glycol-200 hydrogenated glyceryl palmate or a mixture thereof.

18. The isotropic wash composition according to claim 12 wherein the wash composition does not comprise an acrylate-based thickener.

19. The isotropic wash composition according to claim 12 wherein the wash composition further comprises a sunscreen, terpineol, thymol, zinc pyrithione, octopirox or a mixture thereof.

20. The isotropic wash composition according to claim 12 wherein the wash composition comprises less than 5.5% by weight soap, and no more than 2.0% by weight of ethanol, halogenated antimicrobial actives, cyclosilicones, parabens and hydantoins.

21. The isotropic wash composition according to claim 12 wherein the composition further comprises green tea, yarrow, ginseng, marigold, hibiscus, ginko biloba, chamomile, licorice, aloe vera, grape seed, citrus unshiu, willow bark, sage, rosemary, bambusa vulgaris shoot extract or a mixture thereof.

22. The isotropic wash composition according to claim 12 wherein the wash composition is an anti-acne face wash composition, the anionic surfactant comprises 95 to 100% by weight taurate based on total weight of the anionic surfactant, the zwitterionic surfactant comprises 95 to 100% by weight betaine based on total weight of zwitterionic surfactant, the taurate and betaine are at a weight ratio from 1:3 to 3:1 and the wash composition has total surfactant that does not exceed 15% by weight of the wash composition.

23. The isotropic wash composition according to claim 12 wherein the wash composition comprises less than 25 ppm 1,4 dioxane.

24. The isotropic wash composition according to claim 12 wherein the wash composition further comprises a fragrance, vicinal diol or both.

25. The isotropic wash composition according to claim 12 wherein the wash composition further comprises vitamin B₂, vitamin B₃, vitamin B₆, vitamin C, vitamin E, folic Acid, biotin, salt or a mixture thereof.

26. The wash composition according to claim 12 wherein the composition further comprises a ceremide, pseudoceramides or a mixture thereof.
